(19) United States
(12) Patent Application Publication
(10) Pub. No.: US 2011/0257506 A1
(43) Pub. Date: Oct. 20, 2011

(54) NON-INVASIVE METHOD AND SYSTEM FOR DETECTING AND EVALUATING NEURAL ELECTROPHYSIOLOGICAL ACTIVITY

(75) Inventors: Sylvain Baillet, Shorewood, WI (US); Line Garnero, Clamart (FR); Didier Berthoumieux, Clamart (FR); Florence Gombert, Paris (FR)

(73) Assignee: Centre National De La Recherche Scientifique(CNRS)

(21) Appl. No.: 12/988,827

(22) PCT Filed: Apr. 23, 2009
(86) PCT No.: PCT/FR09/00483

§ 371 Date: May 27, 2011

(30) Foreign Application Priority Data
Apr. 24, 2008 (FR) 0802305

(51) Int. Cl. A61B 5/055; A61B 5/0476

(52) U.S. Cl. 600/410; 600/544

(57) ABSTRACT

The disclosure pertains to a non-invasive method and system for detecting and evaluating neural electrophysiological sources by exploring a multiplicity of points belonging to a zone of interest. The non-invasive techniques pose problems as to the instability of the estimation in relation to the position of the measurement points and errors of geometrical registration with complementary anatomical examinations, this possibly generating significant errors. The present disclosure is aimed at proposing a non-invasive method and system for detecting and evaluating profound neural electrophysiological activity which is both fast, complete and accurate. In this regard, the disclosure is aimed at a non-invasive method of detecting and evaluating neural electrophysiological activity comprising a step of non-invasive acquisition of anatomical and electrophysiological data in an analysis region, a step of identifying at least one electrophysiological source and a step of selecting at least one main measurement point, characterized in that it furthermore comprises a step of estimating the electrical potentials at a plurality of secondary measurement points belonging to a zone of interest situated around the main measurement point.

### CROSS-REFERENCE TO RELATED APPLICATIONS

This application is a National Phase Entry of International Application No. PCT/FR2009/000483, filed on Apr. 23, 2009, which claims priority to French application Ser. No. 08/02305, filed on Apr. 24, 2008, both of which are incorporated by reference herein.

### TECHNICAL FIELD

The invention relates to a non invasive method and system for detecting and evaluating neural electrophysiological sources by exploring a plurality of points belonging to an area of interest. The present invention pertains to the field of electrophysiological signal acquisition and processing, via non invasive cerebral imaging, particularly, in the frame of therapeutic decision support and medical diagnosis. More particularly, the invention relates to a non invasive method and system for detecting and evaluating the neural activity in subjects suffering from neurological, distinctive electrophysiological signature, diseases such as epilepsy, neurodegenerative diseases, Alzheimer's disease, Parkinson's disease, etc.

### BACKGROUND

The analysis of cerebral electrophysiological signals aims at identifying the cerebral areas involved in normal or pathological electrophysiological activity. Various tools are known from the related art which make it possible to collect and analyze electric signals corresponding to the neural electrophysiological activity of a subject.

The first one consists in implanting invasive intracranial electrodes, for example, within regions which may exhibit epileptogenicity. This type of implantation requires a delicate, risky surgery and possibly traumatic for patients. In fact, it consists in placing in a highly precise manner electrodes in the brain so as to record the activity of regions suspected to have a pathologic electrophysiological activity. The risk of infections and of subdural hematomas associated to the implantation is high. The subject remains implanted for long observation time periods in the specialized clinical services and the economical costs relating to this type of protocol are considerable. Moreover, in certain instances the implantation does not allow to identify with certainty the cerebral regions to be treated because the spatial sampling allowed by this technique is limited to a few hundreds of measurement points in the cerebral volume.

Other, non invasive methods do exist which make use of electroencephalography (EEG) or magnetoencephalography (MEG) techniques and make it possible to obtain a suitable spatial resolution (centimeter) for the functional study of the brain. These surface observation techniques require, following the acquisition of the electrophysiological signals, the use of mathematical tools which make it possible, through resolution of the direct problem and the inverse problem, to locate and reconstruct from the surface observations acquired in certain points, the cerebral electric activity generated within an area of interest which may extend to the entire brain. These techniques have the advantage of exhibiting an excellent time resolution while making it possible to analyze the neural electrophysiological phenomenoa without surgery. Such a method is particularly described in patent document FR 2 893 434.

However, these non invasive techniques also exhibit certain technical issues relating to the matching between the detection of the neural activities and their precise anatomic origin. In fact, the imaging technique requires the registration of the MEG or EEG recordings with a structural image of the cortical anatomy which may be obtained at a later stage thanks to an MRI (Magnetic Resonance Imaging) examination. This operation includes numerous sources of errors and inaccuracies (in the order of the centimeter at the most). Yet, small variations of the relative position of the measurement points with respect to the targeted anatomic area of interest lead to high variations of the neural current estimation corresponding thereto.

More particularly, during these non invasive measurements, the effects of these drawbacks consist in generating inaccuracies and a numerical instability pertaining to the various mathematical models used upstream. Thus, it is not possible to obtain, through these estimation methods results reliable enough to avoid, in this clinical context, intracranial electrode analysis.

### SUMMARY

The present invention aims at overcoming the drawbacks of the related art by providing a non invasive method and system for the detection and the evaluation of the neural electrophysiological activity which are fast, thorough and accurate. Another object aims at providing the clinician with reliable and representative information of the cerebral activity within the environment of a region of interest so as to integrate the variability of the results in its final diagnosis. To this end, the invention provides a step of estimating electrophysiological potentials within a region of interest located around a predetermined anatomic target so as to integrate the uncertainty over the measurements due to errors of relative repositioning of the cortical anatomy and MEG or EEG surface recordings.

More particularly, the object of the invention is a non invasive method for detecting and evaluating the neural electrophysiological activity comprising a step of acquiring anatomic and electrophysiological data in a non invasive manner within an analysis region, a step of identifying at least one electrophysiological source and a step of selecting at least a main measurement point. This method further comprises a step of estimating electric potentials at a plurality of secondary measurement points belonging to an area of interest located around the main measurement point. Thus, measurement instability related to the main measurement point positioning is compensated by the plurality of secondary measurement points which make it possible to obtain a source and a module for selecting at least one main measurement point, further including, a module for estimating electric potentials at a plurality of secondary measurement points belonging to an area of interest located around the main measurement point. According to particular features:
the module for estimating electric potentials includes means for classifying the secondary measurement points (52) according to two classes;
the module for estimating the electric potentials includes means for calculating electrophysiological potentials representing each of the classes.

### BRIEF DESCRIPTION OF THE DRAWINGS

Other features and advantages of the invention will become more apparent from the reading of the following detailed embodiment, with reference to the accompanying figures which respectively represent:

FIG. 1 is a schematic representation of an embodiment of a system for the implementation of the neural electrophysiological activity detection and evaluation method according to the invention;

FIG. 2 is a flowchart of the method according to the invention;

FIG. 3 is a numerical representation of a cortex portion and of an area of interest; and

FIGS. 4a, 4b, 4c and 4d, four charts representing the electrophysiological signals measured by intracranial electrodes or estimated through the method according to the invention.

### DETAILED DESCRIPTION OF AN EMBODIMENT

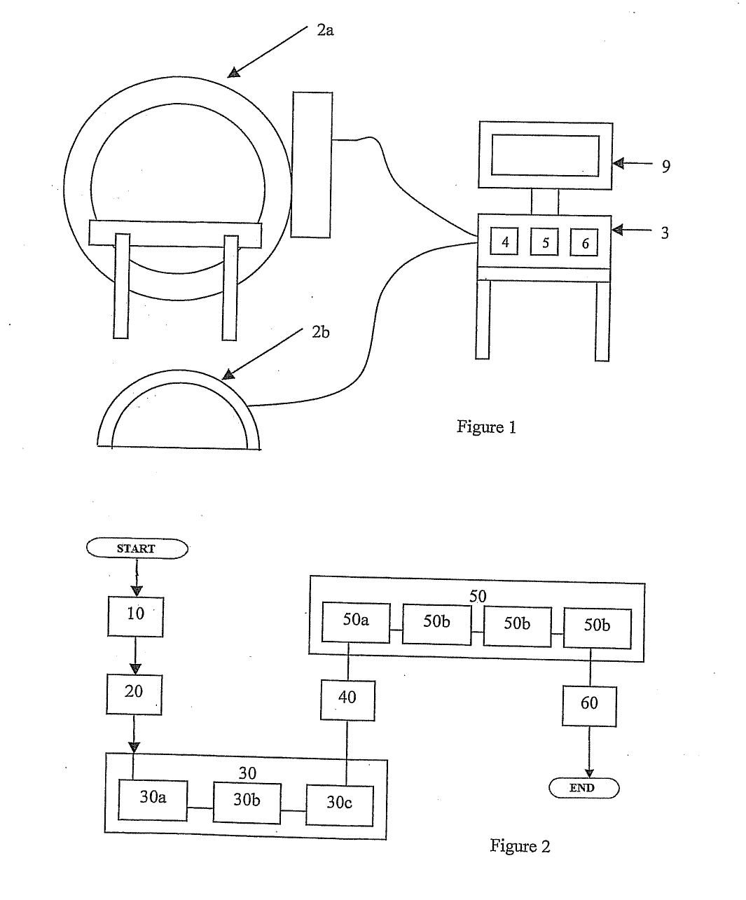

An embodiment of a system for the implementation of the neural electrophysiological activity detection and evaluation method according to the invention will now be described with reference to the flowchart of FIG. 1. The system comprises a magnetic resonance imaging apparatus 2a, hereafter MRI, as well as a magnetoencephalograph 2b, hereafter MEG, for the acquisition of electrophysiological data. These two apparatuses 2a and 2b are connected to a processing unit 3 composed of a module 4 for resolving the direct problem, a module 5 for resolving the inverse problem over the entire meshing of the cortex and a module 6 for estimating the electrophysiological potentials within an area of interest 8. The processing unit is further advantageously connected to a display device 9 for representing the electrophysiological signals obtained through the method of the invention.

The neural electrophysiological activity detection and evaluation method illustrated in FIG. 2 includes a first step 10 of acquiring physiological data for modelizing an analysis region 12, for example the entire cerebral cortex of a subject. This modelization step is carried out through the anatomic MRI 2a weighed in T1. Data are stored and the MRI examination of the subject is segmented so as to constitute a surface meshing of the cerebral thorough estimation of deep signals representative of the environment within the area of interest.

According to particular features:
the selection step consists in selecting the implantation of virtual electrodes, defining the main measurement points, according to the electrophysiological data acquired in the preceding steps;
the estimation step includes a phase of classifying of the secondary measurement points in particular according to the electrophysiological data acquired during the preceding steps. This classification is advantageous in that it makes it possible to provide the user with two different signals which are thus representative of the environment of the area of interest;
the classification is further made through singular value decomposition;
the classification is made through a classification to the nearest neighbor in the meaning of the K-mean algorithm;
the method includes a phase of calculating the electrophysiological potentials representing each of the classes;
the area of interest substantially corresponds to a cube of 1 cm³ centered on the main measurement point.

The invention also relates to a non invasive system for detecting and evaluating the neural electrophysiological activity comprising apparatuses for the acquisition of anatomic and electrophysiological data within an analysis region, a module for identifying at least one electrophysiological cortex. Moreover, three first markers, such as vitamin A chips are placed on the skull of the subject before the MRI so as to reposition the head with the MEG 2b system for subsequent treatment.

The second step 20 of the method according to the invention consists in carrying out a magnetoencephalographic examination of the subject. This MEG examination consists in acquiring and scanning the surface electromagnetic data collected using a MEG 2a apparatus composed of a plurality of sensors positioned on the cortical surface of the subject. According to a preferred embodiment, the magnetoencephalographic examination is carried out through a CTF/VSM MedTech MEG system, the number of MEG sensors being equal to 151 and the sampling rate being equal to 1250 Hz. Alternatively, any recording made on an equivalent MEG instrument, or even an EEG system incorporating a plurality of scalp electrodes may be subject to the analysis proposed by the invention.

The EEG or MEG examination consists in recording the cerebral activity of the subject either at rest, with eyes open or closed, or during an experimental paradigm for exploring certain particular functions of the brain such as perception, language, memory, attentiveness, etc. the duration of the recording should be sufficient for ensuring the acquisition of at least one electrophysiological event of interest for the study, in this case at least an epileptic spike. Three second markers such as coils located at the same positions as for the MRI, for instance, on the nasion, left ear and right ear, make it possible to mark the position of the MEG sensors with respect to the anatomy of the subject.

The third step 30 of the method according to the invention consists in identifying the electrophysiological sources of the analyzed region. First of all, in a first phase 30a of the method consists in a registration of the data from both MRI 2a and MEG 2b measurement systems. This registration is made by superposing first and second markers. Alternatively, there are registration systems with a higher number of guide marks using a complete scanning of the scalp by an Isotrak/Polhemus type 3-D positioning system, or an equivalent system.

Then, the electrophysiological data recorded during the first 10 and second 20 step are used during a phase of resolving the direct problem 30b. Thus, the direct problem resolution module 4 makes it possible to modelize the potentials and magnetic fields collected from the scalp and generated by a known source configuration. It provides a gain matrix mathematically linking the sources to the MEG sensors. Advantageously, this problem may be resolved with the MEG/EEG data visualization and processing BrainStorm software (see for example web site http://neuroimage.usc.edu/brainstorm/).

A third phase 30c of step 30 for estimating the position of the electrophysiological sources, consists in, in accordance with the direct model, reconstructing and identifying in time and space, the generators, or electrophysiological sources, at the origin of the electrophysiological signals collected on surface by the MEG 2b system. This step 30c, achieved by the inverse problem resolution module 5, makes it possible to identify the electrophysiological sources of signals recorded outside the head by the MEG sensors. This technique for resolving the inverse problem is particularly described in document: “S. Baillet, J. C. Mosher, R. M. Leahy, “electromagnetic brain imaging”, IEE Signal Proc. Mag. 18(6), 14-30, November 2001”. This problem may advantageously arise when the sources are to be detected on the surface of the cortex obtained by processing the MRI examination of the subject according to step 10 and following the relative repositioning of the functional MEG or EEG MRI and anatomic information according to step 30a.

For example, according to a particular embodiment, it is possible to use said standard minimal weighed method for identifying the configuration of neural sources of cortical origin whereof the global energy is minimal among all the configurations modelizing the MEG/EEG surface data in an equivalent manner. In MEG, the modelization of the direct problem is written as follows:

B=GJ+ε, where
B is the data matrix containing the MEG or EEG surface measurements whereof the number of rows corresponds to the number of sensors and whereof the number of columns corresponds to the number of time samples of the recordings;
G is the gain matrix which is given by the direct problem according to the procedure of step 30b;
J is the unknown matrix of the cortical sources of which the respective amplitudes are sought to be estimated; and
E represents the noise present in the recordings.

Many methods for estimating the cortical source matrix J from the data matrix B containing the MEG or EEG surface measurements and of gain matrix G have been published to date but a small number of them turned out to be practicable on real physiological recordings, the latter containing noise and disturbances rendering fragile the most sophisticated estimation methods. The estimation of the cortical sources matrix J may advantageously be carried out according to the very general principle of regularized estimation whereof the principle, in the case of the estimator of the weighed minimal standard as well as in step 30c, consisting in minimizing a function of the cortical source matrix J of type:

∥B−GJ∥²+λ∥J∥²; where
∥B−GJ∥² represents the gap between the measurements and their model produced by the cortical source matrix J via the gain matrix G;
λ∥J∥² ensures the regularity of the reconstruction and the robustness to disturbances present in the measurements; and
The term λ is a parameter which weighs the regularizing term with respect to the adjustment of the model to the data.
   The advantage of minimizing this error is that the cortical source matrix J estimation problem has a unique solution of analytical form, which may thus be explicitly calculated.

Other calculation methods for calculating the direct problem are presented in publication:

Mosher, J. C.; Leahy, R. M. & Lewis, P. S. EEG and MEG: forward solutions for inverse methods; IEEE Trans Biomed Eng, 1999, 46, 245-259. Moreover, with regard to the contribution of the geometrical registration between MRI and MEG or EEG, it is also possible to refer to the publication;

Dale A, Sereno M (1993) Improved localization of cortical activity by combining EEG and MEG with MRI surface reconstruction: a linear approach. J. cognitive Neuroscience 5, 162-176.

By the end of these three first steps 10, 20 and 30, the processing unit has identified the cortical origin electrophysiological sources of recordings MEG or EEG. Then, during a fourth step 40, the method according to the invention consists in allowing the investigator to select the position of the main measurement points 42 whereof the electric potentials created by the corresponding neural electrophysiological sources are estimated. Advantageously, this technical aspect allows the investigator to access a virtual electrode implantation scheme 44 of a depth comprising at least one virtual sensor corresponding to a main measurement point 42. In fact, the method proposes the visualization on the display screen 9, of the electrophysiological data acquired during steps 10, 20 and 30 and to visualize the electrophysiological activities collected according to the virtual depth electrode implantation 44.

In the clinical context, the position of the main measurement points 42 may be determined according to the usual clinical workup of the subject which leads to the elaboration of a depth electrode implantation scheme. Thus, the regions liable to be at the origin of a pathological cerebral activity prioritarily targeted by the clinician and will be subjected to electrode virtual implantation according to the principles of the invention. In the context of the exploration of a healthy brain, the investigator may determine the anatomic localization of regions exhibiting an interest presumably in the context of the subject of the experimental study (occipital cortex and vision, hippocampus and memory, etc.).

The uncertainties relating to the experimental handling, and more particularly those due to errors of relative positioning of the functional MEG or EEG and MRI anatomic data acquired separately may cause strong variations, which presumably are not well controlled, of the estimation of the electrophysiological potentials at each main measurement point 42. Thus, the method according to the invention hence provides a step of estimating the electrophysiological potentials 50 at a plurality of secondary measurement points 52 covering an area of interest 8 around the main measurement point 42 and whereof the dimensions cover the uncertainties relating to the geometrical registration between the MEG/EEG and MRI examinations.

According to a non limiting embodiment, the area of interest 8 corresponds to a cube of a 1 cm side centered at the main measurement point 42 and the internal volume of this area of interest 8 is sampled at 1000 secondary measurement points 52. However, according to an alternative embodiment, the dimensions of the area of interest 8 and the sampling in this area of interest 8 may be directly defined by the investigator. The dimensions of the area of interest 8 are linked to both the repositioning uncertainty between the functional MEG/EEG and MRI anatomic examinations and to the distance between two consecutive measurement points such as defined by the investigator. In a clinical environment, and if for example it is about simulating deep electrode implantation in a subject, the volume of the area of interest may be limited by the distance separating two consecutive electrodes for the material which will in fine be used by the neurosurgeon during the surgery.

However, the larger the volume of the area of interest, the weaker the consistency of the measurements within this volume because they will be much less representative of the uncertainty regarding the neural current estimation at a particular point of the cortex. Contrarily, a too small area will not make it possible to correctly manage the measurement of uncertainties relating to a particular estimation of the neural currents. Moreover, the dimensions of the area of interest are the consequence of a compromise between the consistency of the measurements and the level of the measurement of the uncertainties on the particular estimation. Thus, it is possible to estimate that the area of interest 8 may be advantageously represented by a 1 cm side cube, thus, easily encompassing the afore-mentioned geometrical registration uncertainties.

The method comprises a first phase 50a of estimating the electrophysiological potentials at each one of the secondary measurement points 52 and a second phase 50b of allotting the estimated electrophysiological potentials within the area of interest 8 according to two different and antagonist classes so as to provide the clinicians with two different signals which are representative of the environment within the area of interest 8 and which incorporate the variation of the results inherent to the experimental context of the measurements. The method according to the invention thus, makes it possible to establish a highly reliable estimation compared to a method presenting a single signal.

According to another preferred embodiment of the invention, the classification is carried out according to a singular value decomposition, and a classification to the nearest neighbors through the K-mean method (kmeans). The singular value decomposition is a mathematical method which consists in decomposing a measurement matrix M over bases of orthonormal vectors, called singular vectors, on the left U and on the right V weighed by singular values arranged on the diagonal of a singular matrix S, such that M=U·S·V′, where V′ is the transposed matrix of V.

In an alternative approach, an independent component analysis is used. Here, the singular value decomposition is used in order to update tendencies in the spatial distribution of the electrophysiological potentials at each one of the secondary measurement points 52 within the area of interest 8. If this area of interest 8 is a 1 cm side cube, it may be decomposed into 1000 secondary measurement points 52. Thus, each row of the measurement matrix M is composed of evolution of time of one of these secondary potentials 52. The number of columns of the measurement matrix M corresponds to the number of time samples specific to the collected data.

Following the decomposition of the measurement matrix M, the singular vectors within matrix U represent an orthonormal time series basis, thus, correlated. Hence, the corresponding singular values denote the contributions in terms of relative power among all the original measurements. Then, the method consists in the recovery of the first two components of matrix U exhibiting the highest relative powers and multiplying them by the respective two first singular values S, so as to extract the most representative two measurements of matrix M.

Then, the method consists in calculating the time correlation rate between:
the matrix U component exhibiting the highest relative power, and thus, the most representative, of all the measurements of matrix M; and
the time series of the secondary measurement points of matrix M.
   The same method is applied for the second component of matrix U. The two time series of matrix M exhibiting the maximum correlation rate with the first and second component are then extracted. These two time series are for initializing a step 52b of classifying time series of measurement matrix M according to two classes so as to provide a compact representation of the variability of the measurements within the predefined area of interest 8.

According to a preferred method of the invention, the time series classification is carried out according to the kmeans principle, preferably with k equal to two classes. The time series classification may alternatively be carried out with any time series classification approach. The measurement used to classify the time series of measurement matrix M is based on the time correlation between the measurement series and the two classes series.

Once the time series of measurement matrix M classified according to any one of both classes, singular value decomposition is applied again to the time series of the measurements of each class. Thus, two evolutions over time representing the variability of the original measurements are exhibited within the area of interest 8. Advantageously, during a step 60, the method then aims at representing both signals corresponding to the electrophysiological potentials representative of each class on the display screen, such that the investigator may consider the instability and the variability of the results in the experimental measurement analysis.

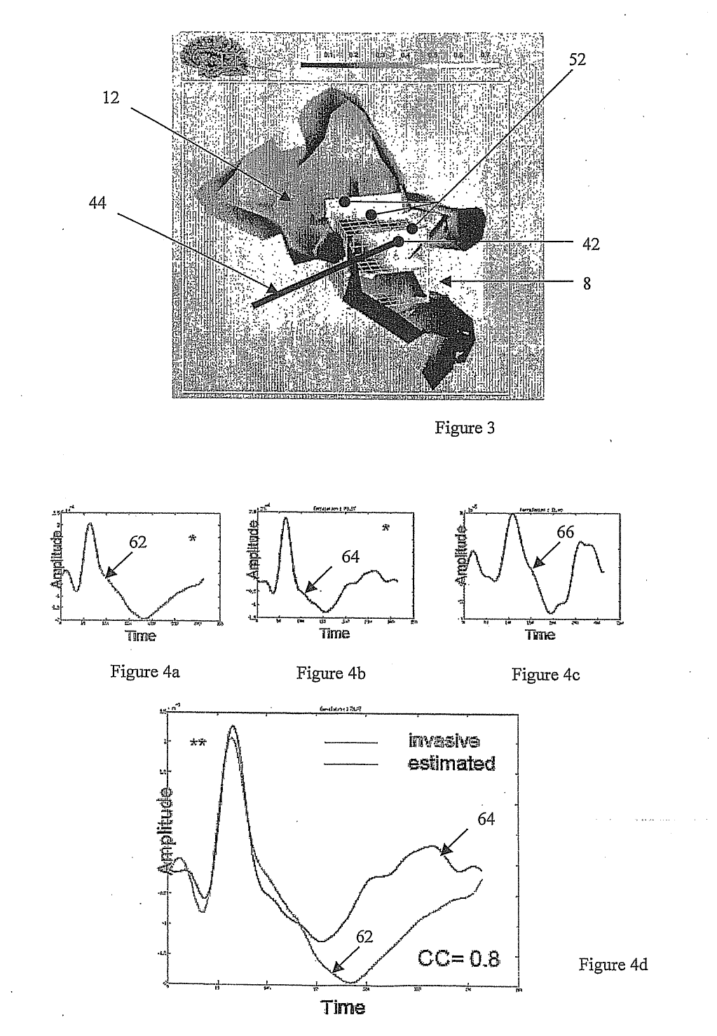

FIG. 3 represents a portion of the cortex and an area of interest 8, in this case a 1 cm side cube centered at the main measurement point 42 defined by a depth virtual electrode 44. It is worth observing the correlation of the potentials estimated within this area of interest 8 with respect to the original deep signal measured using a real intracranial electrode. Two areas of distinct colors clearly appear: a first area whereof the activity is weakly correlated to the real measure (dark colors) and a second area which is highly correlated (light colors).

Experimental results obtained on a subject suffering from a form of epilepsy show a good estimation of the “spike” type signals which characterize the epileptic syndrome. These results are illustrated by FIGS. 4a, 4b, 4c and 4d. FIG. 4a represents the electrophysiological potential 62 measured by an invasive intracranial electrode at a main measurement point, whereas FIGS. 4b and 4c represent the electrophysiological potentials 64 and 66 estimated within an area of interest 8. It is worth noting on FIG. 4d, representing a superposition of the measured signal 62 of FIG. 4a with the estimated signal 64 of FIG. 4b, that the striking events are always detected and the amplitudes of the invasive and estimated signals match. These results have been confirmed on a greater scale, on several subjects.

The invention is not limited to the embodiments described and represented. It is also possible to provide several electrophysiological data acquisition steps before the registration of these data. Moreover, the geometry of the area of interest 8 may be different from the one exhibited.

According to an alternative, the geometry of the area of interest 8 may possibly take into account physiological data acquired during the first step 10 of the MRI. Although the use of the MEG data has been more particularly described, the invention is also applicable, as a matter of principle, to the EEG data analysis.

1. A non-invasive method for detecting and evaluating neural electrophysiological activity, the method comprising a step of acquiring electrophysiological and anatomic data within an analysis region in an non-invasive manner, a step of identifying at least one electrophysiological source and a step of selecting at least a main measurement point, a step of estimating electric potentials at a plurality of secondary measurement points belonging to an area of interest located around the main measurement point.

2. A method according to claim 1, wherein the selection step includes selecting the implantation of virtual electrodes defining the main measurement points, particularly based on the electrophysiological data acquired during the preceding steps.

3. A method according to claim 1, wherein the estimation step includes a phase of classifying the secondary measurement points based on the electrophysiological data acquired during the preceding steps.

4. A method according to claim 3, wherein the classification is carried out by singular value decomposition.

5. A method according to claim 3, wherein the classification is carried out by nearest neighbor classification in the meaning of the K-means algorithm.

6. A method according to claim 1, including a phase of calculating electrophysiological potentials representative of each of the classes.

7. A method according to claim 1, wherein the area of interest substantially corresponds to a cube of 1 cm³ centered on the main measurement point.

8. A non-invasive system for detecting and evaluating neural electrophysiological activity further comprising at least one apparatus of: a magnetic resonance imaging apparatus and a magnetoencephalograph apparatus, operably acquiring electrophysiological and anatomic data within an analysis region, a module for identifying at least one electrophysiological source and a module for selecting at least one main measurement point, a module operably estimating electric potentials at a plurality of secondary measurement points belonging to an area of interest located around the main measurement point.

9. A system according to claim 8, wherein the electric potential estimation module includes means for classifying the secondary measurement points in two classes.

10. A system according to claim 9, wherein the electric potential estimation module includes means for calculating the electrophysiological potentials representative of each one of the classes.
